(19) United States
(12) Patent Application Publication
(10) Pub. No.: US 2022/0105247 A1
(43) Pub. Date: Apr. 7, 2022

(54) BIODEGRADABLE SCAFFOLD FOR HAIR GROWTH AND METHODS OF USE THEREFOR

(71) Applicant: Sanford Burnham Prebys Medical Discovery Institute, La Jolla, CA (US)

(72) Inventors: Alexey V. TERSKIKH, Solana Beach, CA (US); Antonella PINTO, San Diego, CA (US)

(21) Appl. No.: 17/426,869

(22) PCT Filed: Mar. 13, 2020
(86) PCT No.: PCT/US2020/022770

§ 371 Date: Jul. 29, 2021

Related U.S. Application Data
(60) Provisional application No. 62/818,584, filed on Mar. 14, 2019.

(51) Int. Cl. A61L 27/56; C12N 5/071; A61L 27/38; A61L 27/58; A61K 35/36

(52) U.S. Cl. CPC A61L 27/56; C12N 5/0627; A61L 27/3869; A61L 27/58; A61L 27/3834; A61L 2430/18; C12N 2513/00; C12N 2533/40; C12N 2535/00; C12N 2539/00; C12N 2506/45; A61K 35/36

(57) ABSTRACT

Described herein are cellular scaffolds comprising a cell reservoir, a guide attached to the cell reservoir constructed from one or more biodegradable polymers, and a population of folliculo-genic cells. The cellular scaffolds are useful in growing hair.

### CROSS-REFERENCE TO RELATED APPLICATIONS

This application claims the benefit of U.S. Provisional Patent Application No. 62/818,584 filed on Mar. 14, 2019. Priority is claimed pursuant to 35 U.S.C. § 119. The above noted patent application is incorporated by reference as if set forth fully herein.

### BACKGROUND

Folliculogenic-cell related therapies may provide benefit for treatment of hair loss and/or a number of diseases, including alopecias, ectodermal dysplasias, monilethrix, Netherton syndrome, Menkes disease, and hereditary epidermolysis bullosa. Hair loss is associated with diminished self-esteem, emotional distress, secondary morbidity, depressive episodes, and reduced quality of life.

However, replacement of folliculogenic cells requires control and direction of the cells. Additionally, deriving dermal papilla cells from stem cells such as iPSCs has been challenging. Therefore, there remains a need for effective control of the transplantation and growth of folliculogenic cells. There is also a need for improved approaches for implanting folliculogenic cells.

### SUMMARY

The present disclosure aims to meet one or more of the needs identified herein, provide other benefits, or at least provide the public with a useful choice. Certain embodiments provide a cellular scaffold that contains one or more folliculogenic cells in a cell reservoir and further comprises a guide comprising one or more first biodegradable polymers, wherein the guide is attached to the reservoir. Following implantation, the scaffold can function to orient the one or more folliculogenic cells and/or facilitate their development into a functioning hair follicle. The combination of the reservoir and the attached guide may provide improved efficacy relative to previous approaches

In some embodiments, disclosed herein are cellular scaffolds comprising a cell reservoir, a guide attached to the cell reservoir comprising one or more first biodegradable polymers; and one or more folliculogenic cells. The cellular scaffolds may be configured to be implantable in a human patient under the skin. In some aspects the cellular scaffolds include a cell reservoir with a guide extending therefrom. The cellular scaffolds may be configured such that when implanted, the cell reservoir is under the skin while the guide extending therefrom extends outside of the skin. In some embodiments, the cellular scaffolds disclosed herein comprise one or more folliculogenic cells in contact with the cell reservoir. In some embodiments, the cellular scaffolds disclosed herein comprise a cell reservoir that is spherical, ellipsoidal, cylindrical, tubular, or buckyball-shaped. In some aspects, the cell reservoir is configured to enclose or house one or more folliculogenic cells. In this way, such folliculogenic cells may be substantially contained within a location inside of the cell reservoir and under the skin of a patient. In some aspects, the cell reservoir includes one or more anchors or barbs extending from a generally spherical center shell. Such anchors or barbs may further house and secure folliculogenic cells in a secure location under the skin of a patient.

In some embodiments, the cellular scaffolds disclosed herein comprise one or more first biodegradable polymers comprising poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof. In some embodiments, the cellular scaffolds disclosed herein comprise one or more biodegradable polymers comprising polycaprolactone (PCL). In some embodiments, the cellular scaffolds disclosed herein comprise one or more second biodegradable polymers. In some embodiments, the cellular scaffolds disclosed herein comprise one or more second biodegradable polymers comprising poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof. In some embodiments, the cellular scaffolds disclosed herein comprise one or more second biodegradable polymers comprising polycaprolactone (PCL). In some embodiments, the cellular scaffolds disclosed herein comprise one or more first biodegradable polymers and one or more second biodegradable polymers that are the same. In some embodiments, the cellular scaffolds disclosed herein comprise one or more first biodegradable polymers and one or more second biodegradable polymers that are different. In some embodiments, the cellular scaffolds disclosed herein comprise one or more first biodegradable polymers with a slower degradation rate than one or more second biodegradable polymers. In some embodiments, the cellular scaffolds disclosed herein comprise a cell reservoir derivatized with magnetic material. In some embodiments, the cellular scaffolds disclosed herein comprise a guide derivatized with magnetic material. In some embodiments, the cellular scaffolds disclosed herein comprise a cell reservoir 50 to 500 μm in diameter. In some embodiments, the cellular scaffolds disclosed herein comprise a cell reservoir 250-400 μm in diameter, such as 250-350 μm in diameter or 300-400 μm in diameter. In some embodiments, the cellular scaffolds disclosed herein comprise a cell reservoir about 300 μm in diameter. In some embodiments, the cellular scaffolds disclosed herein comprise a cell reservoir about 200 μm in diameter. In some embodiments, the cellular scaffolds disclosed herein comprise a hollow guide. In some embodiments, the cellular scaffolds disclosed herein comprise a solid guide.

In some embodiments, the cellular scaffolds disclosed herein comprise one or more folliculogenic cells and the one or more folliculogenic cells are stem cells. In some embodiments, the cellular scaffolds disclosed herein comprise stem cells selected from the group consisting of embryonic stem cells and induced pluripotent stem cells (iPSCs). In some embodiments, the cellular scaffolds disclosed herein comprise induced pluripotent stem cells (iPSCs) generated from a source selected from the group consisting of fibroblast cells, renal epithelial cells, and blood cells. In some embodiments, the cellular scaffolds disclosed herein comprise one or more folliculogenic cells and the one or more folliculogenic cells are dermal papilla, epithelial stem cells, or a combination thereof.

In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue. In some embodiments, the methods of growing a hair follicle disclosed herein comprise piercing a dermal tissue with a needle prior to implanting the cellular scaffold. In some embodiments, the methods of growing a hair follicle disclosed herein comprise loading one or more folliculogenic cells into a cellular scaffold, and the loading comprises placing the one or more folliculogenic cells and the cellular scaffold into a microwell of a microwell plate comprising multiple microwells. In some embodiments, the methods of growing a hair follicle disclosed herein comprise loading one or more folliculogenic cells into a cellular scaffold, and the loading comprises placing the one or more folliculogenic cells and the cellular scaffold into a microwell of a microwell plate comprising multiple microwells, wherein each microwell of the microwell plate comprises one or more folliculogenic cells and a cellular scaffold. In some embodiments, the methods of growing a hair follicle disclosed herein comprise loading one or more folliculogenic cells into a cellular scaffold, and the loading comprises placing the one or more folliculogenic cells and the cellular scaffold into a microwell of a microwell plate comprising multiple microwells, and the cellular scaffold is orientated in the microwell such that the cell reservoir is closer to a bottom of the microwell and the guide is closer to an opening of the microwell. In some embodiments, the methods of growing a hair follicle disclosed herein comprise loading one or more folliculogenic cells into a cellular scaffold, and the loading comprises placing the one or more folliculogenic cells and the cellular scaffold into a microwell of a microwell plate comprising multiple microwells, and the loading one or more folliculogenic cells into a cellular scaffold is performed manually. In some embodiments, the methods of growing a hair follicle disclosed herein comprise loading one or more folliculogenic cells into a cellular scaffold, and the loading comprises placing the one or more folliculogenic cells and the cellular scaffold into a microwell of a microwell plate comprising multiple microwells, and the loading one or more folliculogenic cells into a cellular scaffold is performed robotically. In some embodiments, the methods of growing a hair follicle disclosed herein comprise loading one or more folliculogenic cells into a cellular scaffold, and the cell reservoir, the guide, or both the cell reservoir and the guide are derivatized with magnetic material. In some embodiments, the methods of growing a hair follicle disclosed herein comprise loading one or more folliculogenic cells into a cellular scaffold, and the loading comprises applying a magnetic field to the cellular scaffold. In some embodiments, the methods of growing a hair follicle disclosed herein comprise loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the implanting the cellular scaffold into dermal tissue further comprises applying a magnetic field to the cellular scaffold. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the one or more folliculogenic cells are stem cells at the step of implanting the cellular scaffold into dermal tissue. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and further comprising covering the dermal tissue with a bandage after implanting the cellular scaffold into dermal tissue.

In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the one or more folliculogenic cells are in contact with the cell reservoir. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the cell reservoir is spherical, ellipsoidal, cylindrical, tubular, or buckyball-shaped. The cell reservoir may have a porous exterior and a hollow or porous interior. In some embodiments, the cell reservoir is hollow. In some embodiments, the cell reservoir has a porous interior. For example, a spherical, cylindrical, or ellipsoidal cell reservoir may have a hollow interior or a porous interior. A cell reservoir having a porous interior may have a degree of porosity greater than or equal to 90%, 95%, 97%, 98%, 99%, or 99.5%. The degree of porosity is determined as 100% minus p, where p is the percentage of mass of a non-porous solid object of the same shape, size, and composition as the cell reservoir (thus, a reservoir with 99% porosity would have 1% of the mass of a non-porous solid object of the same shape, size, and composition as the cell reservoir). In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the one or more first biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the one or more first biodegradable polymers comprises polycaprolactone (PCL). In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the cell reservoir comprises one or more second biodegradable polymers. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the cell reservoir comprises one or more second biodegradable polymers comprising poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the cell reservoir comprises one or more second biodegradable polymers comprising polycaprolactone (PCL). In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the cell reservoir comprises one or more second biodegradable polymers, and wherein the one or more first biodegradable polymers and the one or more second biodegradable polymers are the same. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the cell reservoir comprises one or more second biodegradable polymers and wherein the one or more first biodegradable polymers and the one or more second biodegradable polymers are different. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the cell reservoir comprises one or more second biodegradable polymers, and wherein the one or more first biodegradable polymers has a slower degradation rate than the one or more second biodegradable polymers. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the cell reservoir is derivatized with magnetic material. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the guide is derivatized with magnetic material. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the cell reservoir is 50 to 500 μm in diameter. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the cell reservoir is about 200 μm in diameter. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the guide is hollow. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the guide is solid.

In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the one or more folliculogenic cells are stem cells. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, and wherein the one or more folliculogenic cells are stem cells selected from the group consisting of embryonic stem cells and induced pluripotent stem cells (iPSCs). In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the one or more folliculogenic cells are stem cells selected from the group consisting of embryonic stem cells and induced pluripotent stem cells (iPSCs), and wherein the induced pluripotent stem cells (iPSCs) are generated from a source selected from the group consisting of fibroblast cells, renal epithelial cells, and blood cells. In some embodiments, disclosed herein are methods of growing a hair follicle comprising loading one or more folliculogenic cells into a cellular scaffold, wherein the cellular scaffold comprises a cell reservoir and a guide attached to the cell reservoir comprising one or more first biodegradable polymers, and implanting the cellular scaffold into dermal tissue, wherein the one or more folliculogenic cells are dermal papilla, epithelial stem cells, or a combination thereof.

In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, and distributing one or more folliculogenic cells over the plurality of microwells of the microwell plate; and incubating the microwell plate. In some embodiments, the microwell plate is subjected to centrifugation after distributing one or more folliculogenic cells over the plurality of microwells of the microwell plate. Such a step can promote bringing the folliculogenic cells into contact with the cell reservoir and/or promote entry of the folliculogenic cells into the cell reservoir. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, and distributing one or more folliculogenic cells over the plurality of microwells of the microwell plate, and wherein the one or more folliculogenic cells are in contact with the cell reservoir. In some embodiments, the plurality of microwells are conical or U-shaped. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, and wherein the cell reservoir is spherical, ellipsoidal, cylindrical, tubular, or buckyball-shaped. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, and wherein the one or more first biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, and wherein the one or more first biodegradable polymers comprises polycaprolactone (PCL). In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers and comprising one or more second biodegradable polymers. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers and comprising one or more second biodegradable polymers, wherein the one or more second biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers and comprising one or more second biodegradable polymers, wherein the one or more second biodegradable polymers comprises polycaprolactone (PCL). In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers and comprising one or more second biodegradable polymers, wherein the one or more first biodegradable polymers and the one or more second biodegradable polymers are the same. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers and comprising one or more second biodegradable polymers, wherein the one or more first biodegradable polymers and the one or more second biodegradable polymers are different. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers and comprising one or more second biodegradable polymers, wherein the one or more first biodegradable polymers has a slower degradation rate than the one or more second biodegradable polymers. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the cell reservoir is derivatized with magnetic material. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the guide is derivatized with magnetic material. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the cell reservoir is 50 to 500 μm in diameter. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the cell reservoir is about 200 μm in diameter. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the guide is hollow. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the guide is solid.

In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the one or more folliculogenic cells are stem cells. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the one or more folliculogenic cells are stem cells and wherein the stem cells are selected from the group consisting of embryonic stem cells and induced pluripotent stem cells (iPSCs). In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the one or more folliculogenic cells are stem cells and wherein the stem cells are selected from the group consisting of embryonic stem cells and induced pluripotent stem cells (iPSCs), wherein the induced pluripotent stem cells (iPSCs) are generated from a source selected from the group consisting of fibroblast cells, renal epithelial cells, and blood cells. In some embodiments, disclosed herein are methods of culturing folliculogenic cells comprising loading one cellular scaffold into each microwell of a microwell plate, wherein the microwell plate comprises a plurality of microwells, and wherein each cellular scaffold comprises a cell reservoir and a guide attached to the cell reservoir comprising one or more first biodegradable polymers, wherein the one or more folliculogenic cells are dermal papilla, epithelial stem cells, or a combination thereof.

In some embodiments, disclosed herein are methods of generating a cellular scaffold comprising utilizing two-photon polymerization to generate a cellular scaffold comprising a cell reservoir and a guide attached to the cell reservoir comprising one or more first biodegradable polymers. In some embodiments, disclosed herein are methods of generating a cellular scaffold comprising utilizing volumetric techniques such as computed axial lithography to generate a cellular scaffold comprising a cell reservoir and a guide attached to the cell reservoir comprising one or more first biodegradable polymers.

In some embodiments, cellular scaffolds disclosed herein are for use in therapy, e.g., in treating hair loss and/or a condition selected from an alopecia, ectodermal dysplasia, monilethrix, Netherton syndrome, Menkes disease, or hereditary epidermolysis bullosa, in a subject in need thereof. Such treatment may comprise the steps of a method of growing a hair follicle described herein.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the compositions and methods disclosed herein are set forth with particularity in the appended claims. A better understanding of the features and advantages of the scaffolds and methods of use thereof will be obtained by reference to the following detailed description that sets forth illustrative embodiments, in which the principles of the disclosed scaffolds and methods are utilized, and the accompanying drawings of which:

FIG. 1 shows a Lolli-up micro-scaffold mechanism of action. FIG. 1A depicts an artist's interpretation of empty lolli-up showing an intended position inside the skin with a guide penetrating the epidermis of the skin. FIG. 1B depicts an artist's interpretation of a lolli-up pre-loaded with epithelial and dermal papilla cells transplanted into the skin with a guide penetrating the epidermis of the skin. FIG. 1C depicts an artist's interpretation of a nascent hair follicle with a degrading lolli-up (dashed lines) pre-loaded with formed dermal papilla capsule (magenta) and epithelial cells (blue) penetrating the epidermis of the skin. FIG. 1D depicts an actual electron microscopy image of an actual PCL lolli-up. Scale bar 500 μM.

FIG. 2A-C shows a bioengineered tool for generating a custom plate for Lolli-up loading. FIG. 2A depicts a negative solid stamp; FIG. 2B depicts a positive silicone mold; and FIG. 2C depicts an agarose microwell dish.

FIG. 3A-B shows additional views of a bioengineered tool for generating a custom plate for Lolli-up loading. FIG. 3A shows a view of the entire plate; FIG. 3B shows a stereo image of the center of the plate showing individual Lolli-up scaffolds distributed into the wells.

FIG. 4 shows Lolli-up loading with human induced pluripotent stem cells (iPSC)-derived dermal papilla cells and mouse E18.5 embryonic epithelial cells. Stereo-microscope images of individual Lolli-up scaffolds at day 0, 1, and 3 after loading with dermal papilla and keratinocytes.

FIG. 5 shows examples of transdermal hair growth after transplantation of Lolli-up scaffolds loaded with human induced pluripotent stem cell-derived dermal papilla cells (iPSC-DP)+mouse E18.5 epithelial cells. Zoomed-in pictures of hair shaft observed in Nude mice at day 28, 40 and 50 after transplantation of folliculogenic cells engineered in the micro-scaffold. The black color of the hair shaft is contributed by the melanocytes present in the C57BL6 epithelial cell preparation.

FIG. 6 shows an example of an improved lolliup unit design. The depicted design has material optimized for bio-degredation time, mechanical properties, and reduced toxicity.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein.

Embodiments of the scaffolds and methods of use provided herein relate to inducing hair growth in a subject. In some embodiments, a population of folliculogenic cells can be generated and combined with a cellular scaffold, which can then be subcutaneously implanted into a subject and generate hair. Advantageously, each population of folliculogenic cells can be amplified to provide a vast population of cells for each cellular scaffold. The combinations of folliculogenic cells and cellular scaffolds can be used to treat subjects having skin disorders, as well as subjects having burns, scars, and/or hair loss.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” The term “or” is used in an inclusive sense, i.e., equivalent to “and/or,” unless the context dictates otherwise. Section divisions in the specification are provided for the convenience of the reader only and do not limit any combination of elements discussed.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All references cited herein are incorporated by reference in their entirety as though fully set forth. In case of any contradiction or conflict between material incorporated by reference and the expressly described content provided herein, the expressly described content controls. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

When indicating the number of substituents, the term “one or more” refers to the range from one substituent to the highest possible number of substitution, e.g. replacement of one hydrogen up to replacement of all hydrogens by substituents.

The term “optional” or “optionally” with respect to an event or circumstance denotes that the subsequently described event or circumstance can but need not occur, and that the description includes instances where the event or circumstance occurs and instances in which it does not. The term “optional” or “optionally” with respect to an element denotes that the subsequently described element can but need not be present, and that the description includes embodiments in which the element is present and embodiments in which it is absent.

The term “folliculogenic cell” as used herein generally refers to a population of cells that function to grow hair follicles.

The term “dermal papillae cell” as used herein generally refers to a unique population of mesenchymal cells that regulate hair follicle formation and the hair growth cycle. During development most dermal papillae (DP) cells are derived from mesoderm, however, functionally equivalent DP cells of cephalic hairs can originate from Neural Crest (NC).

The term “cellular scaffold” as used herein refers to a structure comprising a solid material (e.g., polymeric) that is capable of containing cells, where “solid” is used in the sense of state of matter, e.g., in contrast to liquid; the scaffold may be porous, as discussed elsewhere herein. Hydrogels, e.g., collagen or agarose hydrogels, are not considered solid and do not qualify as a solid material of a cellular scaffold. The solid material may be a biodegradable polymer, as described in detail elsewhere herein.

The term “attached” and grammatical variants thereof as used herein denotes that a first element is adhered, connected, or joined to a second element. Mere contact without more does not constitute attachment.

The terms “administer,” “administering”, “administration,” and the like, as used herein, refer to delivering scaffolds or compositions to the desired site of biological action. Appropriate delivery methods include, but are not limited to parenteral routes (including subcutaneous (s.c.), intraperitoneal (i.p.), and topical (top.) administration). Those of skill in the art are familiar with administration techniques that can be employed with the scaffolds and methods described herein. In some embodiments, the scaffolds are administered subcutaneoulsy.

The terms “co-administration” or the like, as used herein, are meant to encompass administration of the selected therapeutic agents to a single patient, and are intended to include treatment regimens in which the agents are administered by the same or different route of administration, in the same or different composition, or at the same or different time.

The terms “effective amount” or “therapeutically effective amount,” as used herein, refer to a sufficient amount of an agent or a composition being administered which will relieve to some extent one or more of the symptoms of the disease or condition being treated; for example a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “preventing” or “prevention” of a disease state denotes causing the clinical symptoms of the disease state not to develop in a subject that can be exposed to or predisposed to the disease state, but does not yet experience or display symptoms of the disease state.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

### Biodegradable Microscaffolds for Facilitating Cell Transplantation and Hair Growth

Disclosed herein are cellular scaffolds, which may be biodegradable, comprising a cell reservoir (including reservoirs shaped as a buckyball and any other spherical structures) and a biodegradable guide attached to the cell reservoir. In some embodiments, the cell reservoir or the guide is derivatized with magnetic materials to facilitate handling and manipulations. In some embodiments, the cell reservoir and guide scaffold (referred to herein as a “Lolli-up” or “lolliup”) serves to facilitate transplantation of folliculogenic cells into the skin. The Lolli-ups disclosed herein are transplantation ready and are loaded with single or multiple cell types. In some embodiments, the Lolli-ups disclosed herein are loaded with folliculogenic cells, and folliculogenic cells may comprise dermal papilla, epithelial cells or combinations thereof in any given ratio. Additional cell types such as melanocytes, endothelial cells, or neural/neuronal cells, can be added to the constructs. In some of the methods disclosed herein, a cellular scaffold is pre-loaded with cells and then transplanted into the skin. In some embodiments, the cell reservoir of the scaffold is located in the dermis, while the guide penetrates out and through the thickness of the epidermis. The guide may serve to facilitate the hair shaft penetration through the skin and more particularly, through the skin epidermis. Additionally, in some embodiments, the guide can maintain a temporary opening in the epidermis to enable the hair shaft penetration through the skin.

In some embodiments, the Lolli-up scaffolds disclosed herein are loaded with cells by depositing the scaffold into a microwell (which may be custom-fabricated) which enables vertical positioning of the Lolli-up scaffold with the guide at the top and the cell reservoir at the bottom. In some embodiments, a single cell suspension is added into the microwell containing the lolliup scaffold and then the plate or manifold comprising the microwells is spun to collect the cells at the bottom of the well and inside the scaffold. In some embodiments, parallel loading of multiple lolliups is accomplished, e.g., by using an agarose multiwell dish, which may be custom-printed. The systems and methods of parallel loading of multiple lolliups can be automated and is easily manipulated by a robotic device. In some aspects, multiple lolliups may be connected to one another with one or more cross members to provide additional support. The cross members may be biodegradable. In some aspects, an array of interconnected lolliups is prepared. In some aspects, the lolliups are positioned at an angle with respect to the exterior surface of the skin such that new hair follicles may be eventually directed at an angle with respect to the surface of the skin to provide a more natural look.

In certain embodiments, the methods of this disclosure comprise administering to a subject a scaffold comprising a cell reservoir and a guide and one or more folliculogenic cells. In some embodiments, the methods provide a therapy including, for example, hair growth. The administration can be to any location of a subject's skin, for example, a location of the subject's skin such scalp, face, upper lip, chin, eyebrow, eyelash, arm, leg, back, torso, hand, foot, and abdomen. In some embodiments, the location of the subject's skin includes scar tissue. In some embodiments, the subject has alopecia. In some embodiments, the subject is mammalian, such as a human.

In some embodiments, the scaffolds are administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. In some embodiments, the composition is administered over a period of at least 1 week, at least 2 weeks, at least 3 weeks, at least 4 weeks, at least 6 weeks, at least 7 weeks, at least 8 weeks, at least 9 weeks, at least 10 weeks, 11, at least 12 weeks, at least 24 weeks, at least 48 weeks, or at least 52 weeks or longer. In some embodiments, the scaffolds are administered once daily, twice daily, once every week, once every two weeks, once every three weeks, once every four weeks (or once a month), once every 8 weeks (or once every 2 months), once every 12 weeks (or once every 3 months), or once every 24 weeks (once every 6 months).

In some embodiments, the cellular scaffolds disclosed herein are generated via two-photon polymerization. In some embodiments, the cellular scaffolds disclosed herein are generated using different volumetric techniques, including computed axial lithography. In some embodiments, the cellular scaffolds disclosed herein are generated using computed axial lithography, including the methods discussed in Kelly et al., Volumetric additive manufacturing via tomographic reconstruction, SCIENCE 363(6431): 1075-1079 (2019).

In some embodiments, the methods of this disclosure comprise administering to a subject multiple Lolli-up scaffolds simultaneously into multiple sites in the skin such that the spacing between each individual Lolli-up scaffold is approximately the same as the spacing between the hair follicles in normal skin. Hair grown from the Lolli-up scaffolds administered in this manner have a natural, cosmetically appealing look. In some embodiments, at least 10, 20, 30, 40, 50, 60, 70, 80, 90, or 100 individual Lolli-up scaffolds are administered. In some embodiments, at least 110, 120, 130, 140, 150, 160, 170, 180, 190, 200, 210, 220, 230, 240, 250, 260, 270, 280, 290, or 300 individual Lolli-up scaffolds are administered. In some embodiments, at least 325, 350, 375, 400, 425, 450, 475, 500, 525, 550, 575, 600, 625, 650, 675, 700, 725, 750, 775, 800, 825, 850, 875, 900, 925, 950, 975, or 1000 individual Lolli-up scaffolds are administered. In some embodiments, at least 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 individual Lolli-up scaffolds are administered. In some embodiments, each individual Lolli-up scaffold is not attached to another individual Lolli-up scaffold. In some embodiments, one or more individual Lolli-up scaffold is attached to one or more additional individual Lolli-up scaffolds. In some embodiments, an individual Lolli-up scaffold is attached to one other Lolli-up scaffold. In some embodiments, an individual Lolli-up scaffold is attached to two other Lolli-up scaffolds. In some embodiments, an individual Lolli-up scaffold is attached to three other Lolli-up scaffolds. In some embodiments, an individual Lolli-up scaffold is attached to four other Lolli-up scaffolds. In some embodiments, an individual Lolli-up scaffold is attached to four other Lolli-up scaffolds and the plurality of Lolli-up scaffolds form a grid pattern. In some embodiments, an individual Lolli-up scaffold is attached to four other Lolli-up scaffolds and the plurality of Lolli-up scaffolds form an array of Loll-up scaffolds.

In some embodiments, the Lolli-up scaffolds disclosed herein are administered to a subject so that the guide forms a 90° angle with the subject's skin. In some embodiments, the Lolli-up scaffolds disclosed herein are administered to a subject so that the guide forms an 80° angle with the subject's skin. In some embodiments, the Lolli-up scaffolds disclosed herein are administered to a subject so that the guide forms a 70° angle with the subject's skin. In some embodiments, the Lolli-up scaffolds disclosed herein are administered to a subject so that the guide forms a 60° angle with the subject's skin. In some embodiments, the Lolli-up scaffolds disclosed herein are administered to a subject so that the guide forms an angle with the subject's skin that is less than 60°, less than 50°, less than 40°, less than 30°, less than 20°, or less than 10°. In some embodiments, the Lolli-up scaffolds disclosed herein are administered to a subject so that hair exiting the guide creates a naturally occurring angle with the subject's skin. In some embodiments, the Lolli-up scaffolds disclosed herein are administered to a subject so that hair exiting the guide matches the angle of the natural hair adjacent to the Lolli-up scaffold in the subject's skin.

In some embodiments, the Lolli-up scaffolds disclosed herein comprise a cell reservoir and a guide and one or more folliculogenic cells, and the guide extends into the cell reservoir. In some embodiments, the Lolli-up scaffolds disclosed herein comprise a cell reservoir and a guide and one or more folliculogenic cells, and the guide does not extend into the cell reservoir. In some embodiments, the Lolli-up scaffolds disclosed herein comprise a cell reservoir and a guide and one or more folliculogenic cells, and the guide is bendable. In some embodiments, the Lolli-up scaffolds disclosed herein comprise a cell reservoir and a guide and one or more folliculogenic cells, and the guide is resistant to cracking. In some embodiments, the Lolli-up scaffolds disclosed herein comprise a cell reservoir and a guide and one or more folliculogenic cells, and the Lolli-up scaffolds are administered to a subject so that the guide extends through the subject's skin and the cell reservoir is anchored entirely beneath the subject's skin. In some embodiments, the Lolli-up scaffolds disclosed herein comprise a cell reservoir and a guide and one or more folliculogenic cells, and the Lolli-up scaffolds are administered to a subject so that at least a portion of the guide extends out through the subject's skin and the cell reservoir is anchored entirely beneath the subject's skin. In some embodiments, the Lolli-up scaffolds disclosed herein comprise a cell reservoir and a guide and one or more folliculogenic cells, and the Lolli-up scaffolds are administered to a subject so that all of the guide extends out through the subject's skin and the cell reservoir is anchored entirely beneath the subject's skin.

### Figure Descriptions

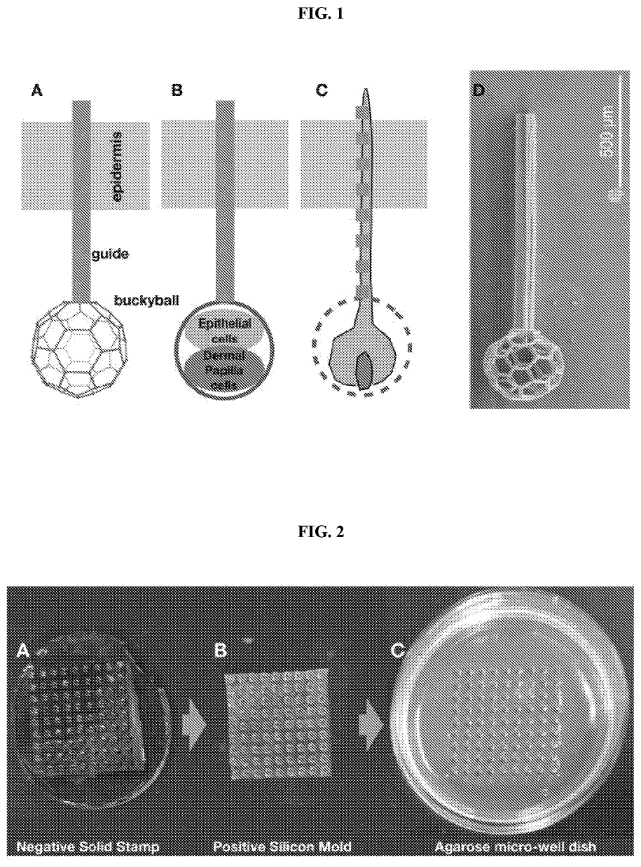

FIGS. 1A-1C are illustrative schematics showing the Lolli-up micro-scaffold suggested mechanism of action according to one embodiment. More particularly, FIG. 1A illustrates a schematics of an empty lolliup showing an intended position inside the skin with a guide extending therefrom and penetrating the epidermis of the skin. FIG. 1B is a schematic showing a lolliup pre-loaded with epithelial and dermal papilla cells and transplanted into the skin with a guide extending therefrom and penetrating the epidermis of the skin. FIG. 1C is a schematic showing a nascent hair follicle with a degrading lolliup (dashed lines) pre-loaded with formed dermal papilla capsule (magenta) and epithelial cells (blue) penetrating the epidermis of the skin. FIG. 1D is an electron microscopy image of an exemplary PCL lolliup. Scale bar 500 μM.

FIG. 2 Shows a Bioengineered tool for generating a custom plate for Lolli-up loading according to one embodiment. (A) negative solid stamp, (B) positive silicone mold. (C) agarose microwell dish.

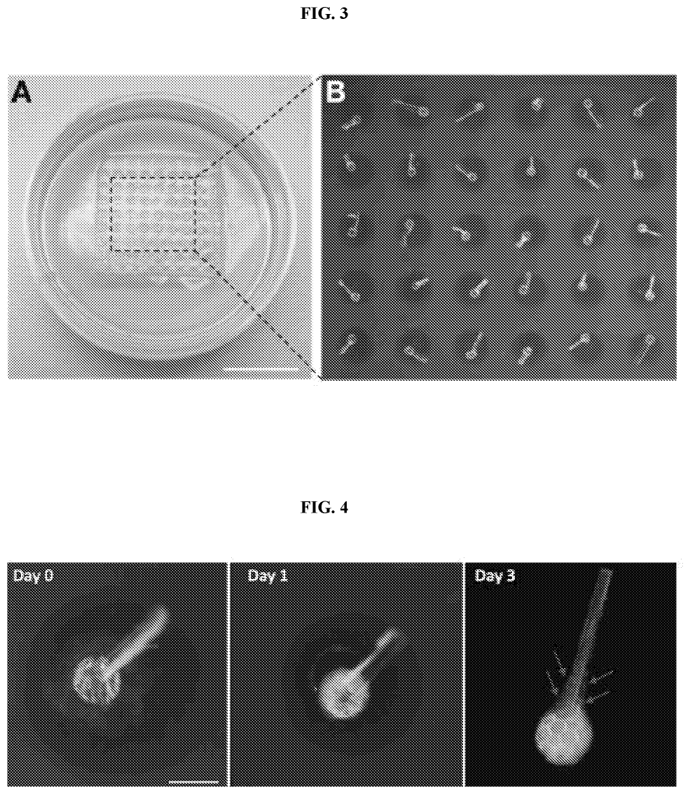

FIG. 3 Shows a Bioengineered tool for generating a custom plate for Lolli-up loading according to one embodiment. Agarose microwell holding lolliup scaffolds placed individually into each resection. (A) view of the entire plate, (B) stereo image of the center of the plate showing individual lolliup scaffolds distributed into the wells.

FIG. 4 Shows a Lolli-up loading with human iPSC-derived dermal papilla cells and mouse E18.5 embryonic epithelial cells according to one embodiment. Stereo-microscope images of individual lolliup scaffolds at day 0 (zero), 1 (one), and 3 (three) after loading with dermal papilla and keratinocytes. Note the cell migration along the shaft in day 3 image. Day 0 and 1 were taken inside the microwell. Day 3 image were taken with the construct removed from the microwell and placed on the bottom of a petri dish prior to transplantation into Nude mice. Note the cells migrating along the guide away from the buckyball (Day 3, red arrows). Images were taken using a stereo-microscope. Scale bar: 100 μm.

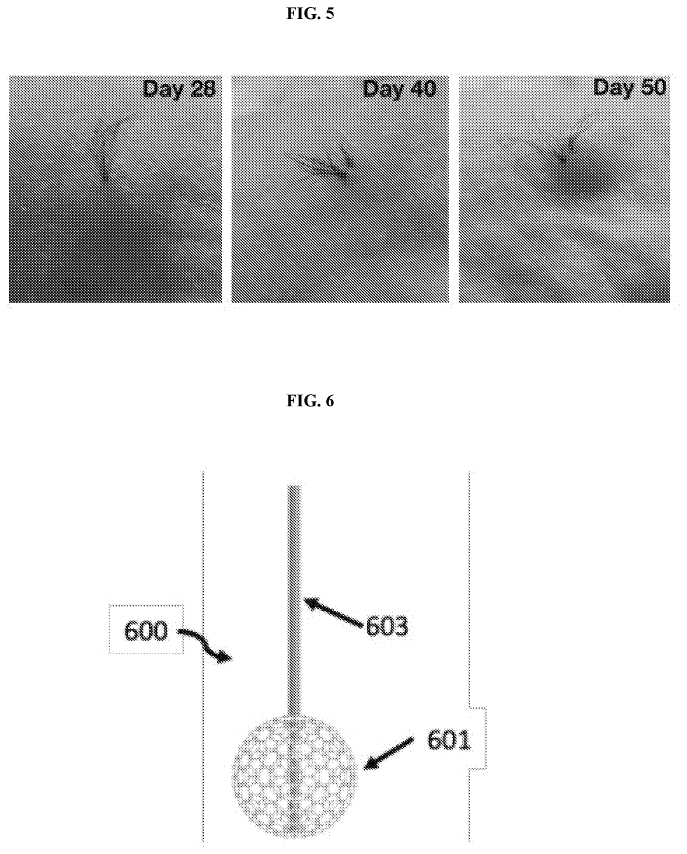

FIG. 5 Shows an Example of transdermal hair growth after transplantation of lolli-up scaffolds loaded with human iPSC-DP+mouse E18.5 epithelial cells according to one embodiment. Zoomed-in images of hair shaft observed in Nude mice at day 28, 40 and 50 after transplantation of folliculogenic cells engineered in the micro-scaffold. The black color of the hair shaft is contributed by the melanocytes present in the C57BL6 epithelial cell preparation.

FIG. 6 depicts another exemplary embodiment of Lolli-up micro-scaffold. As shown, the Lolli-up 600 may include a cell reservoir 601 and a guide 603. The cell reservoir 601 may be substantially spherical and may be configured to house folliculogenic cells within the cell reservoir 601. The cell reservoir 601 may be porous such that the folliculogenic cells may be substantially contained within the cell reservoir 601 yet may also be able to migrate outside of the cell reservoir 601 over time. The cell reservoir 601 may be biodegradable over time. The guide 603 may be attached to the cell reservoir 601 or may be part of a unitary structure that comprises the Lolli-up 600. As shown in FIG. 6, at least a portion of the guide 603 may extend into the cell reservoir 601. In some aspects the guide 603 may extend through the length of the cell reservoir 601. In some aspects, the guide 603 may be secured to the top and the bottom of the cell reservoir 601. Extending the guide 603 through the cell reservoir 601 may improve the structural integrity of the Lolli-up 600.

The Lolli-up 600 may be configured such that the guide 603 extends through the skin when the Lolli-up 600 is implanted in a patient. In some aspects the guide 603 is not hollow but rather a solid member or thread. In this way, while the guide 603 extends through the skin, the skin substantially surrounds the guide 603 such that the skin maintains its natural ability to prevent intrusion of harmful materials into the body of the patient. In some aspects the guide 603 comprises a nylon thread. In some aspects, the guide is made of a biodegradable material such that it degrades over time. In the guide 603 is made of a biodegradable polyester such as polycaprolactone (PCL). In general, the guide 603 is configured to guide the cells upward. In some aspects, after implantation of the Lolli-up 600, one or more hair shafts may coalesce at that skin-guide 603 interface.

While preferred embodiments have been shown and described herein, those skilled in the art will understand that such embodiments are provided by way of example only. It should be understood that various alternatives to the embodiments described herein may be employed in practicing the compositions and methods described herein.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1A

### Lolliup Principles and Manufacturing

Lolliup microscaffolds were designed to control the transplantation of folliculogenic cells into skin. The Lolliup consists of a buckyball connected to a guide (shaft). The buckyball houses a given number of cells and is easily manipulated by handling the guide/shaft. In addition to facilitating the handling of cells and the entire lolliup scaffold, the guide/shaft plays a role in holding the space in the skin epidermis for the nascent hair shaft to penetrate and reside during hair growth and hair cycling. Finally, lolliup also set the initial position of hair follicle inside the skin and the direction of hair growth. The lolliup combines the ability to achieve a high cell density (inside a buckyball) with mechanical stability and directionality. Further, loading the lolliup microscaffolds with induced pluripotent stem cell (iPSC)-derived dermal papillae (DP) cells and epithelial cells (including iPSC-derived epithelial cells), enhances the crosstalk between dermal and epidermal cells upon transplantation.

The scaffolds provide an important tool to facilitate the transplantation of the 3D-follicular units into the mouse (and human) skin and to guide the hair growth through the skin, mimicking the biological event of hair follicle formation, as shown in FIG. 1. The buckyball diameter can range from 50 to 500 μM and the guide length can range from 1 to 5 mm. Lolliup microscaffolds have been engineered using two-photon polymerization using polycaprolactone (PCL), the same material that is used for sutures and stiches. Other polymers with biodegradation kinetics and safety profiles well-studied in humans could be used. The buckyball and the guide could be made of different polymers with different degradation kinetics.

### Example 1B

### Manufacturing of Agarose Microwell Dish

A solid stamp was designed to fit 9×9 wells on a 30 mm circle. Sylgard 184 Silicone Elastomer and Curing Agent (purchased from Sigma-Aldrich/Merck KGaA, Darmstadt, Germany) and used as received, was used to produce a silicone positive of the stamp. This positive was then used for agarose molds. A solution of 2% Agarose (A9539-500G, Sigma-Aldrich/Merck KGaA, Darmstadt, Germany) in molecular biology grade water was heated using a microwave until the material was completely dissolved. The liquid agarose was then poured into a 35 mm culture dish. The silicone negative was placed up-side-down on the agarose solution. After 10 minutes, the stamp could be removed from agarose. The result is a culture dish with 63 V-shape microwells, as shown in FIG. 2. The micro-molded agarose culture dish was then transferred in the incubator and equilibrated overnight with the cell medium

### Example 1C

### Distribution of Lolliup Scaffolds Into the Agarose Microwells

To ensure the presence of a single scaffold per microwell and allow spheroids formation into the structure, the Lolli-ups were manually transferred to each microwell. To control that the Lolli-up were placed correctly, with the ball portion on the bottom and the rod toward the top of the resection, the transfer was conducted under a stereomicroscope, as shown in FIG. 3.

### Example 2

### Lolliup Loading with Cells

After differentiation of hiPSCs into DP-like cells and expansion in monolayer, hiPSC-DP cells were harvested with trypsin, centrifuged and resuspended in AmnioMAX II medium (Gibco, Thermo Fischer Scientific, Waltham, Massachusetts, USA) at the density of 1×10⁶ cells in 500 μl. Mouse fetal (E18.5) epithelial cells (keratinocytes) were obtained as described in Zheng et al, 2005 (Ying Zheng, Xiabing Du, Wei Wang, Marylene Boucher, Satish Parimoo, and Kurt S. Stenn, Organogenesis From Dissociated Cells: Generation of Mature Cycling Hair Follicles From Skin-Derived Cells. J Invest Dermatol 124:867-876, 2005). Briefly, the truncal skin was removed from E18.5 C57BL/6 embryos and rinsed in Ca2+ and Mg2+ free PBS. The skin was incubated with 0.2% dispase overnight at 4° C. to allow separation of the epidermis from the dermis. The dermis was then digested with 0.2% collagenase (Sigma-Aldrich, St.Louis, USA) at 37° C. for 60 min, while the epidermis was digested with 0.25% trypsin/EDTA (Sigma-Aldrich, St.Louis, USA) for 10 min at 37° C. Single-cell suspensions were strained through 40 μm filters and pelleted at 1200 rpm. 1×10⁶ single epidermal cell were resuspended in a total volume of 500 μl of Keratinocytes growth medium (PromoCell GmbH, Heidelberg, Germany) while mouse 1×10⁶ single dermal cells were resuspended in AmnioMAX II medium. Single-cell suspensions of 8×10⁵ human iPSC-derived dermal papilla cells or mouse dermal cells were mixed with 8×10⁵ mouse E18.5 epithelial cells and the final volume of medium adjusted to 1 ml. The cells were homogeneously distributed over the microwell in the culture dish.

To ensure a proper settlement of the cell mix at the bottom of the resections, the culture dish was centrifuged for 3 minutes at 500 rpm. At this point, 1 ml of fresh medium made of an equal amount of AmnioMAX™ II and keratinocytes growth medium, was carefully added to the culture dish, which was incubated at 37° C. for up to 3 days. Lolli-up loading with human iPSC-derived dermal papilla cells and mouse E18.5 e3mbryonic epithelial cells is shown in FIG. 4.

### Example 3

### Transplantation of Lolliup Scaffolds Pre-Loaded with Human iPSC-Derived DP Cells in Combination with Mouse E18.5 Epithelial Cells

Athymic nude mice were obtained from Envigo (Envigo, Placentia, USA). All animal procedures were performed in accordance with the PHS Policy on Humane Care and Use of Laboratory Animals and with the approval of the Sanford Burnham Prebys Medical Discovery Institute IACUC Committee.

Ten lolli-up scaffolds loaded with of human iPSC-derived DP cells in combination with mouse E18.5 epithelial cells were transplanted into the back skin of a Nude mouse. Under anesthesia (isofluorane), a shallow stab wound nearly parallel to the skin surface was made using a 20-G ophthalmic V-lance. The scaffolds were manually transferred inside the wound, which was covered with bandages for about 1 week to allow healing. After recovery from anesthesia, mice were single caged and kept under normal husbandry conditions. The skin sites that had received the injected cells were monitored and imaged with a photo camera for 50 days. Hair growth was assessed at day 28 and day 50 following transplantation, as shown in FIG. 5.

In situ analyses of the skin documented expected stereotypic positions of DP cells occupied by human iPSC-DP within newly generated hair follicles and the DP cells were confirmed to be of human origin by immunofluorescence with a human-cytoplasm-specific antibody (data not shown).

The examples and embodiments described herein are for illustrative purposes only and various modifications or changes suggested to persons skilled in the art are to be included within the spirit and purview of this application and scope of the appended claims.

What is claimed is:

1. A cellular scaffold comprising:
(a) a cell reservoir;
(b) a guide attached to the cell reservoir comprising one or more first biodegradable polymers; and
(c) one or more folliculogenic cells.

2. The cellular scaffold of claim 1, wherein the guide is configured such that, when inserted under the skin, at least a portion of the guide extends outside the skin.

3. The cellular scaffold of claim 1, wherein the one or more folliculogenic cells are in contact with the cell reservoir.

4. The cellular scaffold of any of claims 1-3, wherein the cell reservoir is spherical, ellipsoidal, cylindrical, tubular, or buckyball-shaped.

5. The cellular scaffold of any one of the preceding claims, wherein the cell reservoir has a hollow interior.

6. The cellular scaffold of any one of the preceding claims, wherein the cell reservoir has a porous interior.

7. The cellular scaffold of any one of the preceding claims, wherein the one or more first biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof.

8. The cellular scaffold of any one of the preceding claims, wherein the one or more first biodegradable polymers comprises polycaprolactone (PCL).

9. The cellular scaffold of any one of the preceding claims, wherein the cell reservoir comprises one or more second biodegradable polymers.

10. The cellular scaffold of claim 9, wherein the one or more second biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof.

11. The cellular scaffold of claim 9, wherein the one or more second biodegradable polymers comprises polycaprolactone (PCL).

12. The cellular scaffold of any one of claims 9-11, wherein the one or more first biodegradable polymers and the one or more second biodegradable polymers are the same.

13. The cellular scaffold of any one of claims 9-11, wherein the one or more first biodegradable polymers and the one or more second biodegradable polymers are different.

14. The cellular scaffold of claim 13, wherein the one or more first biodegradable polymers has a slower degradation rate than the one or more second biodegradable polymers.

15. The cellular scaffold of any one of the preceding claims, wherein the cell reservoir is derivatized with magnetic material.

16. The cellular scaffold of any one of the preceding claims, wherein the guide is derivatized with magnetic material.

17. The cellular scaffold of any one of the preceding claims, wherein the cell reservoir is 50 to 500 μm in diameter.

18. The cellular scaffold of any one of the preceding claims, wherein the cell reservoir is 250 to 400 μm in diameter (e.g., 250 to 350 μm in diameter or 300 to 400 μm in diameter).

19. The cellular scaffold of claim 17, wherein the cell reservoir is about 200 μm in diameter.

20. The cellular scaffold of claim 17, wherein the cell reservoir is about 300 μm in diameter.

21. The cellular scaffold of any one of the preceding claims, wherein the guide is hollow.

22. The cellular scaffold of any one of the preceding claims, wherein the guide is solid.

23. The cellular scaffold of any one of the preceding claims, wherein the one or more folliculogenic cells are stem cells.

24. The cellular scaffold of claim 23, wherein the stem cells are selected from the group consisting of embryonic stem cells and induced pluripotent stem cells (iPSCs).

25. The cellular scaffold of claim 24, wherein the induced pluripotent stem cells (iPSCs) are generated from a source selected from the group consisting of fibroblast cells, renal epithelial cells, and blood cells.

26. The cellular scaffold of any one of claims 1-25, wherein the one or more folliculogenic cells are dermal papilla, epithelial stem cells, or a combination thereof

27. A method of growing a hair follicle comprising:
(a) loading one or more folliculogenic cells into a cellular scaffold comprising:
i. a cell reservoir; and
ii. a guide attached to the cell reservoir comprising one or more first biodegradable polymers; and
(b) implanting the cellular scaffold into dermal tissue.

28. The method of claim 27, wherein when implanted in a subject, at least a portion of the guide extends outward and through the skin.

29. The method of claim 27, wherein the loading one or more folliculogenic cells into a cellular scaffold comprises placing the one or more folliculogenic cells and the cellular scaffold into a microwell of a microwell plate comprising multiple microwells.

30. The method of claim 29, wherein the microwell is conical or U-shaped.

31. The method of claim 29 or 30, wherein each microwell of the microwell plate comprises one or more folliculogenic cells and a cellular scaffold.

32. The method of any one of claims 29-31, wherein the cellular scaffold is orientated in the microwell such that the cell reservoir is closer to a bottom of the microwell and the guide is closer to an opening of the microwell.

33. The method of any one of claims 29-32, wherein the step of loading one or more folliculogenic cells into a cellular scaffold further comprises subjecting the microwell plate to centrifugation after placing the one or more folliculogenic cells and the cellular scaffold into the microwell.

34. The method of any one of claims 28-33, wherein the loading one or more folliculogenic cells into a cellular scaffold is performed manually.

35. The method of any one of claims 28-33, wherein the loading one or more folliculogenic cells into a cellular scaffold is performed robotically.

36. A method of growing a hair follicle, comprising implanting the cellular scaffold of any one of claims 1-26 into dermal tissue.

37. The method of any one of claims 27-36, further comprising piercing the dermal tissue with a needle prior to implanting the cellular scaffold.

38. The method of any one of claims 27-37, wherein the cell reservoir, the guide, or both the cell reservoir and the guide are derivatized with magnetic material.

39. The method of claim 38, wherein the loading one or more folliculogenic cells into a cellular scaffold further comprises applying a magnetic field to the cellular scaffold.

40. The method of any one of claim 38 or 39, wherein the implanting the cellular scaffold into dermal tissue further comprises applying a magnetic field to the cellular scaffold.

41. The method of any one of claims 27-40, wherein the one or more folliculogenic cells are stem cells at the step of implanting the cellular scaffold into dermal tissue.

42. The method of any one of claims 27-41, further comprising covering the dermal tissue with a bandage after implanting the cellular scaffold into dermal tissue.

43. The method of any one of claims 27-42, wherein the one or more folliculogenic cells are in contact with the cell reservoir.

44. The method of any one of claims 27-43, wherein the cell reservoir is spherical, ellipsoidal, cylindrical, tubular, or buckyball-shaped.

45. The method of any one of claims 27-44, wherein the one or more first biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof.

46. The method of any one of claims 27-44, wherein the one or more first biodegradable polymers comprises polycaprolactone (PCL).

47. The method of any one of claims 27-46, wherein the cell reservoir comprises one or more second biodegradable polymers.

48. The method of claim 47, wherein the one or more second biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof.

49. The method of claim 48, wherein the one or more second biodegradable polymers comprises polycaprolactone (PCL).

50. The method of any one of claims 47-49, wherein the one or more first biodegradable polymers and the one or more second biodegradable polymers are the same.

51. The method of any one of claims 47-49, wherein the one or more first biodegradable polymers and the one or more second biodegradable polymers are different.

52. The method of claim 51, wherein the one or more first biodegradable polymers has a slower degradation rate than the one or more second biodegradable polymers.

53. The method of any one of claims 27-52, wherein the cell reservoir is derivatized with magnetic material.

54. The method of any one of claims 27-53, wherein the guide is derivatized with magnetic material.

55. The method of any one of claims 27-54, wherein the cell reservoir is 50 to 500 μm in diameter (e.g., 250 to 400 μm in diameter, 250 to 350 μm in diameter, or 300 to 400 μm in diameter).

56. The method of claim 55, wherein the cell reservoir is about 200 μm in diameter or about 300 μm in diameter.

57. The method of any one of claims 27-56, wherein the guide is hollow.

58. The method of any one of claims 27-56, wherein the guide is solid.

59. The method of any one of claims 27-58, wherein the one or more folliculogenic cells are stem cells.

60. The method of claim 59, wherein the stem cells are selected from the group consisting of embryonic stem cells and induced pluripotent stem cells (iPSCs).

61. The method of claim 60, wherein the induced pluripotent stem cells (iPSCs) are generated from a source selected from the group consisting of fibroblast cells, renal epithelial cells, and blood cells.

62. The method of any one of claims 27-61, wherein the one or more folliculogenic cells are dermal papilla, epithelial stem cells, or a combination thereof.

63. A method of culturing folliculogenic cells comprising:
(a) loading one cellular scaffold into each microwell of a microwell plate, wherein the microwell plate comprises a plurality of microwells, and wherein each cellular scaffold comprises:
i. a cell reservoir; and
ii. a guide attached to the cell reservoir comprising one or more first biodegradable polymers;
(b) distributing one or more folliculogenic cells over the plurality of microwells of the microwell plate; and
(c) incubating the microwell plate.

64. The method of claim 63, wherein the one or more folliculogenic cells are in contact with the cell reservoir.

65. The method of claim 63 or 64, wherein the cell reservoir is spherical, ellipsoidal, cylindrical, tubular, or buckyball-shaped.

66. The method of any one of claims 63-65, wherein the cell reservoir has a hollow interior.

67. The method of any one of claims 63-65, wherein the cell reservoir has a porous interior.

68. The method of any one of claims 63-67, wherein the microwells are conical or U-shaped.

69. The method of any one of claims 63-68, further comprising centrifuging the microwell plate after distributing one or more folliculogenic cells over the plurality of microwells of the microwell plate.

70. The method of any one of claims 63-69, wherein the one or more first biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof.

71. The method of any one of claims 63-70, wherein the one or more first biodegradable polymers comprises polycaprolactone (PCL).

72. The method of any one of claims 63-71, wherein the cell reservoir comprises one or more second biodegradable polymers.

73. The method of claim 72, wherein the one or more second biodegradable polymers comprises poly(glycolic acid) (PGA), poly(lactic-co-glycolic) acid (PLGA), poly(lactic acid) (PLA), polycaprolactone (PCL), polyethylene glycol, poly(butylene succinate) (PBS), polyphosphazenes, polyanhydrides, polyphosphoesters, polyurethanes, polycarbonates, and combinations thereof.

74. The method of claim 72, wherein the one or more second biodegradable polymers comprises polycaprolactone (PCL).

75. The method of any one of claims 72-74, wherein the one or more first biodegradable polymers and the one or more second biodegradable polymers are the same.

76. The method of any one of claims 72-74, wherein the one or more first biodegradable polymers and the one or more second biodegradable polymers are different.

77. The method of any one of claims 72-74, wherein the one or more first biodegradable polymers has a slower degradation rate than the one or more second biodegradable polymers.

78. The method of any one of claims 63-77, wherein the cell reservoir is derivatized with magnetic material.

79. The method of any one of claims 63-78, wherein the guide is derivatized with magnetic material.

80. The method of any one of claims 63-79, wherein the cell reservoir is 50 to 500 μm in diameter (e.g., 250 to 400 μm in diameter, 250 to 350 μm in diameter, or 300 to 400 μm in diameter).

81. The method of claim 80, wherein the cell reservoir is about 200 μm in diameter or about 300 μm in diameter.

82. The method of any one of claims 63-81, wherein the guide is hollow.

83. The method of any one of claims 63-81, wherein the guide is solid.

84. The method of any one of claims 63-83, wherein the one or more folliculogenic cells are stem cells.

85. The method of claim 84, wherein the stem cells are selected from the group consisting of embryonic stem cells and induced pluripotent stem cells (iPSCs).

86. The method of claim 85, wherein the induced pluripotent stem cells (iPSCs) are generated from a source selected from the group consisting of fibroblast cells, renal epithelial cells, and blood cells.

87. The method of any one of claims 63-83, wherein the one or more folliculogenic cells are dermal papilla, epithelial stem cells, or a combination thereof.

88. A method of generating a cellular scaffold comprising:
(a) utilizing two-photon polymerization to generate a cellular scaffold comprising:
i. a cell reservoir; and
ii. a guide attached to the cell reservoir comprising one or more first biodegradable polymers.

89. The cellular scaffold of any one of claims 1-26, for use in therapy.

90. The cellular scaffold of any one of claims 1-26, for use in treatment of hair loss and/or a condition selected from an alopecia, ectodermal dysplasia, monilethrix, Netherton syndrome, Menkes disease, or hereditary epidermolysis bullosa, in a subject in need thereof.

91. The cellular scaffold of claim 89 or 90, for use in the method of any one of claims 27-62.
